(19) United States
(12) Patent Application Publication
(10) Pub. No.: US 2014/0298926 A1
(43) Pub. Date: Oct. 9, 2014

(54) BIOLOGICAL SAMPLE STORAGE APPARATUS AND METHOD

(71) Applicant: GE HEALTHCARE UK LIMITED, LITTLE CHALFONT (GB)

(72) Inventors: Michael Kenneth Kenrick, Cardiff (GB); Geraint Seymour, Cardiff (GB)

(73) Assignee: GE HEALTHCARE UK LIMITED, CARDIFF (GB)

(21) Appl. No.: 14/358,918

(22) PCT Filed: Nov. 30, 2012
(86) PCT No.: PCT/EP2012/074126

§ 371 Date: May 16, 2014

(30) Foreign Application Priority Data
Nov. 30, 2011 (GB) 1120626.5

(51) Int. Cl. G01N 35/00; C12Q 1/68

(52) U.S. Cl. CPC G01N 35/00663; C12Q 1/6806
USPC 73/864.91

(57) ABSTRACT

Disclosed is a biological sample holder, a container for storing multiple holders, and an automated holder retrieval system. The sample holder may comprise an elongate generally planar support including a handle end and an opposing biological sample storage end, wherein, a substrate for accepting a biological sample is mounted to the holder at said sample storage end. Optionally the substrate is so mounted within a recess in the surface of the support and the recess has a local surface providing a reaction surface for allowing a sub-sample to be removed from the medium by mechanical action, such as cutting or punching. The container may include an array of wells each well being generally square or rectangular in plan and the holders dimensioned so that they are held diagonally in a respective well. The automated biological sample storage apparatus may include a mechanical handling device, and an associated electronic control capable of controlling the mechanical handing device to pick a predetermined sample holder from an associated well, or to place a predetermined sample holder into an associated well, optionally removing or replacing a container lid where a lid is used.

This invention relates to apparatus and a method for densely storing biological samples, in particular, but not exclusively for automated retrieval of such samples.

Biological samples are collected for various reasons, which include collection of DNA or RNA containing sample material from subjects. Often, such samples do not need to be analysed immediately, but rather stored for later analysis, for example to analyse the specific DNA profile of a subject if they succumb to a predefined disease, for statistical DNA research of populations at a later date or for a DNA ‘bank’ for endangered or rare animals.

In some sample storage arrangements, samples need to be frozen, so as not to degrade the sample. However, technology exists to store biological samples at room temperature, without degradation of DNA, by means of storing the samples on a chemically impregnated paper, for example as sold by Whatman, under the brand ‘FTA’. This paper allows the room temperature storage of biological samples for many years. One characteristic of using FTA paper, is that it needs to be dried.

Large cards on which the sample can be stored have been proposed previously. However, when thousands of such cards are kept, the clean storage and retrieval of the cards becomes a problem. When a card needs to be retrieved, conventionally, it has to be found manually and removed from a pile. This gives rise to the likelihood of contamination. Where cards are stacked together then spacers must be inserted between cards to prevent cross contamination. Also, the large storage card format takes up significant storage space when many cards are stored. In addition the presently available storage cards are not ideally suited to retrieval by automated means.

The inventors of the present system have realised that receiving and storing samples on a narrow substrate is possible, and a novel sample holder is disclosed herein. Further, the inventors have realised that a new storage arrangement is required which can allow drying of the substrate, which can prevent cross contamination, and which allows easy automated placing and/or picking of the sample holder.

According to one aspect, the invention comprises a biological sample holder comprising an elongate generally planar support including a handle end and an opposing biological sample storage end, characterised in that a substrate for accepting and storing a biological sample is mounted to the holder at said sample storage end.

In an embodiment, the substrate is so mounted within a recess in the surface of the support. In an alternative embodiment, the support is divided and substrate is sandwiched between the divided support.

In an embodiment, the recess or divided support has a local surface providing a reaction surface for allowing a sub-sample to be removed from the medium by mechanical action, such as cutting or punching. In an embodiment the surface roughness of said local surface is about 0.01-0.25 microns (μm) Rₐ, for example by finishing a plastics mould with a 800 to 1200 UK grit finish.

Alternatively, the recess or divided support includes a window across which the substrate is located, to promote drying of a biological sample on the medium, and to allow a sub-sample of the medium to be removed by accessing both sides of the substrate.

In an embodiment, the holder has a flattened spatula-like form. Optionally, the width of the holder reduces toward the handle end.

Optionally the support includes one or more of: indicia (e.g. a bar code); a radio frequency transponder; and a writable area.

In a preferred embodiment, the holder is approximately rectangular in cross section, with rounded edges, having a generally uniform thickness of about 1 to 3 mm and a width of about 8 to 10 mm at its widest. Optionally the support is 30 to 60 mm long.

In an embodiment the substrate is sheet material about 25 mm long and about 6 to 7 mm in width

In an embodiment, the medium is fixed to the support and preferably occupies about one third of the length of the support at the sample storage end. In an alternative embodiment, the support includes a short handle end, extending about 5 mm beyond the substrate, suitable for mechanical gripping.

In an embodiment the substrate is a fibre based paper, for example cellulose fibre paper sold under the trade name of FTA, FTA Elute, or 903, all by Whatman Inc or a silica fibre base paper, said paper allowing the long term storage of the sample.

According to a second aspect, the invention comprises a multi-well container for holding, or holding, a plurality of biological sample holders according to the first aspect.

In an embodiment the multi-well plate comprises an array of wells each being generally square or rectangular in plan.

In an embodiment, said holders are supportable lengthwise in a respective well, and the width of the holders is wider than the largest width of the wells, such that each holder is supportable only generally diagonally in its respective well.

Optionally the container includes a lid for protecting samples stored on the holders.

According to a third aspect the invention comprises automated biological sample storage apparatus including a plurality of said biological sample holders supported in a plurality of said containers, a mechanical handling device, and an associated electronic control capable of controlling the mechanical handing device to pick a predetermined sample holder from an associated well, or to place a predetermined sample holder into an associated well, optionally removing or replacing a container lid where a lid is used.

According to a fourth aspect, the invention comprises a method for densely storing biological samples, comprising the flowing steps, in any suitable order; a) providing a plurality of biological sample holders, b) causing a sample to be adsorbed or absorbed by the sample holder for storage thereon; c) providing a container including an array of generally square or rectangular section wells separated by well walls; d) placing only one of the plurality of the holders diagonally, at least partly within one or more of the wells; e) storing the container and holder(s); f) retrieving one or more of the plurality of holders, optionally by automated means.

The invention can be put into effect in numerous ways, embodiments only of which are described below, with reference to the drawings, wherein,

FIG. 1a shows an exploded view of a biological sample holder;

FIG. 1b shows a section through the holder shown in FIG. 1a, at line 1b-1b;

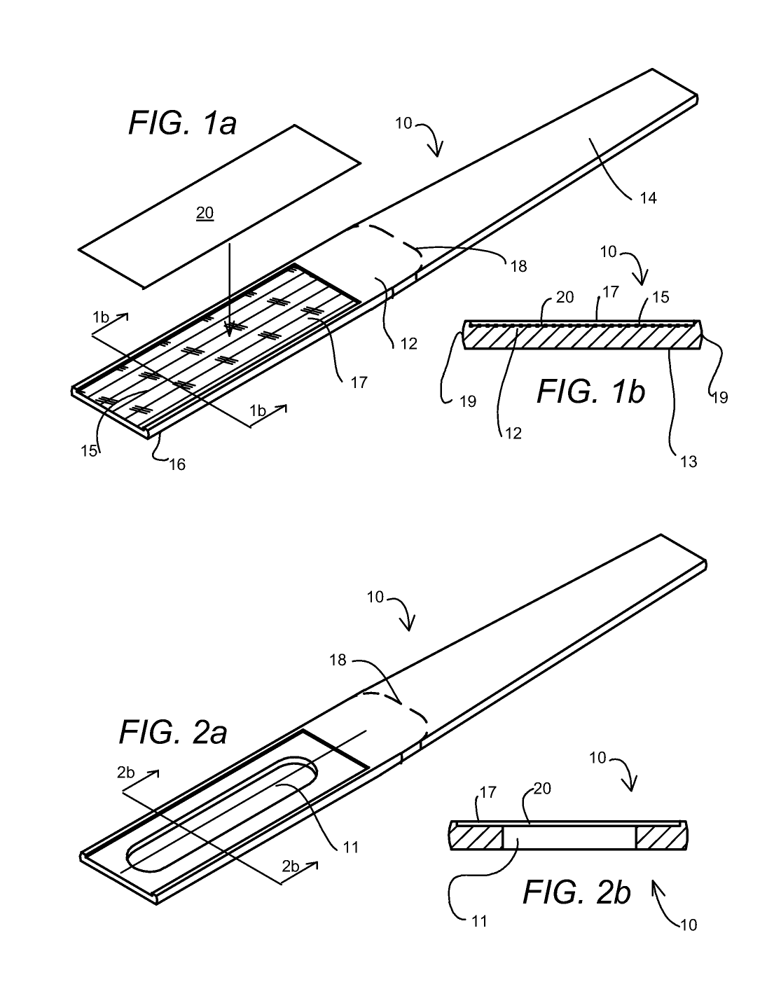

FIG. 2a shows a modified sample holder;

FIG. 2b shows a section through the holder shown in FIG. 2a at line 2b-2b;

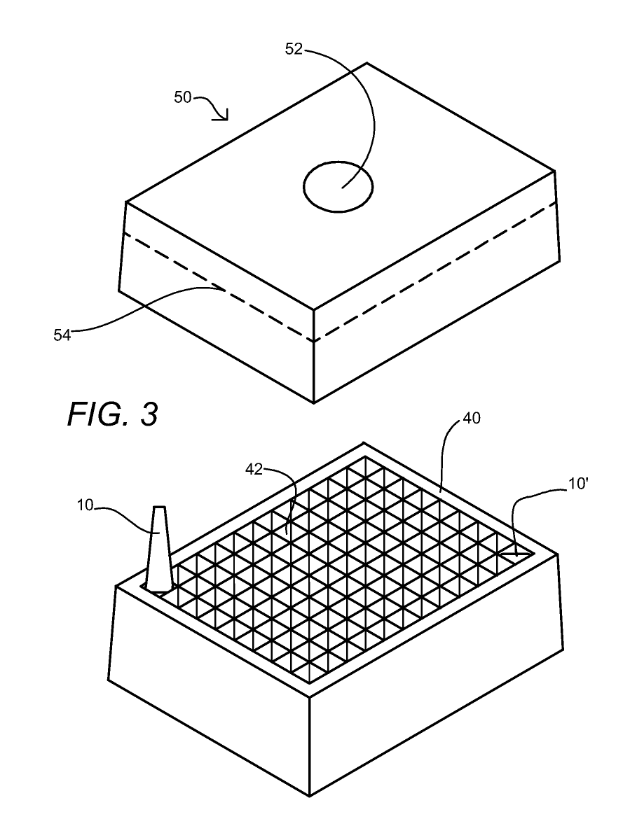

FIG. 3 shows a container for holding the sample holders illustrated in the previous Figures; and

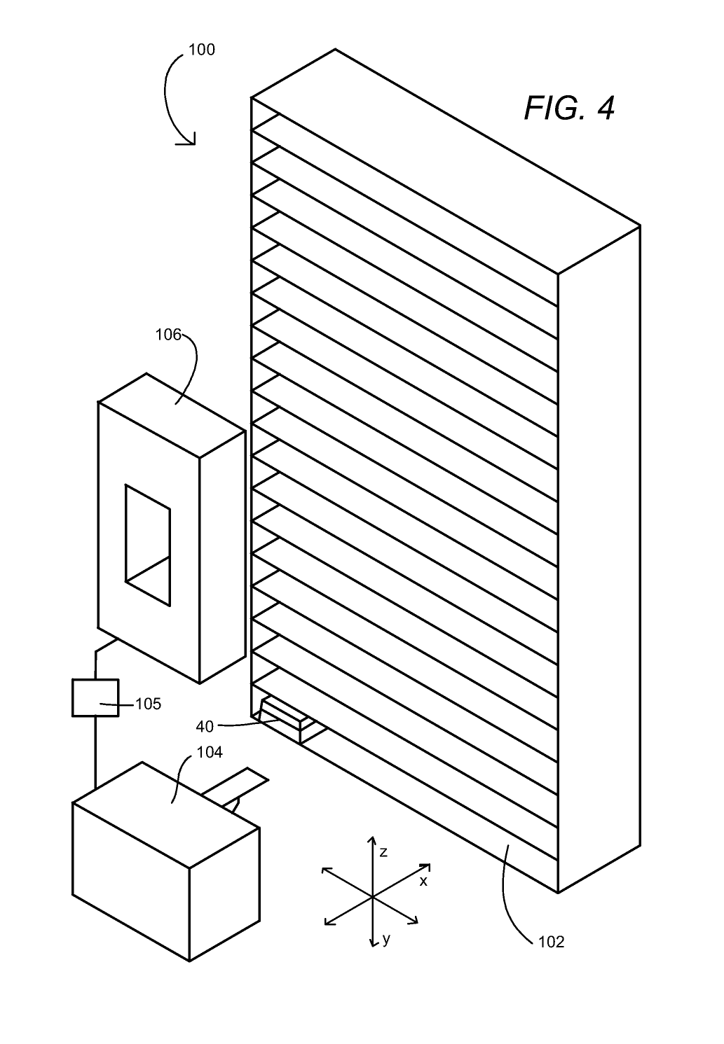

FIG. 4 shows schematically, an automated biological sample storage apparatus, employing holders and containers illustrated in the previous Figures.

FIG. 1a shows a biological sample holder 10 comprising an elongate moulded plastics support 12 having a handle end 14 and a sample storage end 16. A substrate 20, in this case in the form of a sheet of chemically impregnated cellulose fibre paper, sold under the trade name FTA, is shown in exploded detail, but in practice will be attached, for example by means of ultrasonic welding, to the support 12 at the sample storage end 16. To aid mechanical or hand gripping, the handle 14 is tapered toward its distal end.

The storage end 16 includes a shallow recess 17 just deeper than the thickness of the substrate 20. The substrate fits within the recess to keep it in place and protect it during use. In some cases, disks of the substrate material will need to be punched from the substrate, and so a mechanical punch will be needed for that task. The surface 15 of the recess 17 has been roughened to provide a yieldable surface for the punch in use—in other words the surface 15 acts as a backing for cutting a piece of material from the substrate 20.

Referring additionally FIG. 1b, a section of the support 12 is shown with a substrate 20 fitted in place. The Figure shows more clearly the recess 17, its roughened surface 15, and rounded sides 19 of the generally flat rectangular cross section and the planar configuration of the support 12.

In order to permit a different arrangement of holders 10 in a container (described in more detail below), the handle end 14, could end at the chain dotted line 18, defining a short-handled holder 10′ (shown in FIG. 3).

The reverse side 13 of the holder 10 includes a surface on which identifying indicia can be written or placed, for example a bar code or an RF identification tag can be mounted thereon.

Referring to FIGS. 2a and 2b, a modified holder 10 is shown. In this case the recess 17 has a window 11 which encourages drying of the substrate again fitted into the recess 17, and allows access from both sides of the substrate for punching a sub-sample from the substrate.

In use, the substrate will receive a biological sample, such as a blood spot, and the holder 10 can either be put in a sleeve and sent to a central repository where it will be stored, or can be placed directly into a store. In each case the store will be in the form of a container 40 as shown in FIG. 3.

The container 40 is, in this case, a moulded plastics 8 by 12 multi-well plate with a Standard for Bio-molecular Sciences (SBS) standard footprint. In this case, the wells are about 8 mm by 8 mm, in width and about 40 mm deep. Each well 42 has an air hole in its base to allow a little air circulation in each well.

In FIG. 3 a sample holder 10 is shown on the left, and a short-handled holder 10′, as described above, is shown on the right, however, it is expected that the container will be full of holders 10 or 10′. In each case the holders are dimensioned so that they can fit only diagonally in their respective well. Thus the holders are around 9 mm in width and about 3 mm thick.

The container may optionally include a lid 50, which will cover the holders 10 when stored in the container 40. A ventilation hole 52 is used to circulate air or a different gas(es) through the wells where voids are created because the holders are diagonally positioned. Such circulation could be brought about by pressurised gas(es) or a partial vacuum. The gas(es) could be inert or substantially inert, for example nitrogen or carbon dioxide.

Where the short-handled holders 10′ are stored, the lid can be shorter and may finish at the chain dotted line 54. The short-handled holders 10′ allow a close fitting lid to be placed over the wells 42.

In an alternative, the wells 42 can have no holes in their bases, such that the wells can be filled with a gas or gases which are heavier than air, for example carbon dioxide, which will exclude oxygen in the wells and thus help preserve biological material that this stored on the holders 10/10′.

FIG. 4 shows an automated apparatus 100 for storing and retrieving a sample holder 10 or 10′. Additionally, an automated sample processing device 106 is provided to automatically process a retrieved sample.

In practice, a lidded container 40 (along with many others not shown) is placed on a shelving unit 102 by means of a robotic device 104 under control of an electronic control 105, for automatic retrieval. A robotic device 104 can be commanded to retrieve a specific container and place it in the processing machine 106. The processing machine includes further robotic means, for removing the lid of the container 40 and for gripping one of the holders in a well of the container. The gripper passes the holder over a reader to check that the correct holder has been retrieved and the sample on the holder is processed according to known techniques.

Specific embodiments are described above, although alternatives, additions or omissions to those embodiments will be apparent to the skilled addressee. For example, the holders 10 and 10′ and the container 40 are described as manufactured from moulded plastics, however, other materials and manufacture techniques could be employ with equal effect. In order to have a better visualisation of the samples, a transparent plastics is preferred. The substrate 20 is shown held in a recess 17, but other arrangements could be used, for example the support 12 could be divided in two and joined together with the substrate sandwiched therebetween.

The container 40 is shown to have 96 wells, but other numbers of wells could be employed, for example 48, 192 or 384 wells could be used.

Although an automated retrieval system is described and illustrated in FIG. 4, such apparatus is not essential, and a manual retrieval system could be used. Such a manual retrieval system may take the form of the array of handles 14 upstanding from the container 40, or a manually controlled gripper could be employed. Although air circulation has been described, purging the air with, for example, nitrogen, is likely to increase sample viability, and can be achieved by introducing a purging gas either into a sealed storage shelving, or into the containers themselves before sealing the containers, for longer term storage.

The above embodiments provide a simple and effective high density storage system for biological samples, which inherently reduces the risk of cross contamination from handling because a handle or gripper is employed, and of cross contamination from other samples because a physical well wall separates adjacent samples. The samples can be kept dry and ventilated until required. The samples can be readily retrieved by automated means if such a system is employed.

1. A method for densely storing biological samples, comprising the following steps, in any suitable order:
a) providing a plurality of biological sample holders;
b) optionally causing a sample to be adsorbed or absorbed by the sample holder for storage thereon;
c) providing a container including an array of generally square or rectangular section wells separated by well walls;
d) housing one of the holders diagonally, at least partly within at least one of the wells;
e) storing the container and holders; and
f) retrieving one or more of the plurality of holders, optionally by automated means, from a respective well, optionally using the apparatus of claim 13.

2. The method of claim 1, wherein each said biological sample holder comprises an elongate generally planar support including a handle end and an opposing biological sample storage end, wherein a substrate for accepting and storing a biological sample is mounted to the holder at said sample storage end.

3. The method of claim 2, wherein the substrate is so mounted within a recess in the surface of the support.

4. The method of claim 3, wherein the recess has a local surface providing a reaction surface for allowing a sub-sample to be removed from the medium by mechanical action, such as cutting or punching.

5. The method of claim 3, wherein the recess includes a window across which the substrate is located, to promote drying of a biological sample on the medium, and to allow a sub-sample of the medium to be removed by accessing both sides of the substrate.

6. The method of claim 1, wherein the support includes one or more of: indicia (e.g. a bar code); a radio frequency transponder; and a writable area.

7. The method of claim 1, wherein the holder is approximately rectangular in cross section, with rounded edges, having a generally uniform thickness of about 0.5 to 3 mm and a width of about 8 to 10 mm at its widest.

8. The method of claim 1, wherein the substrate is sheet material about 25 mm long and about 6 to 7 mm in width.

9. The method of claim 1, wherein the substrate is a fibre based paper, for example cellulose fibre paper sold under the trade name of FTA, FTA Elute, or 903, all by Whatman Inc or a silica fibre based paper.

10. The method of claim 1, wherein each container is a multi-well container for holding, or holding, a plurality of said biological sample holders.

11. The method of claim 10, wherein said holders are supportable lengthwise in a respective well, and the width of the holders is wider than the largest width of the wells, such that each holder is supportable only generally in said diagonal placement in its respective well.

12. The method of claim 10, wherein each multi-well container includes a lid for protecting samples stored on the holders.

13. An automated biological sample storage apparatus including a plurality of biological sample holders, each supported diagonally in a respective square or rectangular well of a multi-well container, a mechanical handling device, and an associated electronic control capable of controlling the mechanical handing device to pick a predetermined sample holder from a respective well, or to place a predetermined sample holder into a respective well, optionally removing or replacing a container lid where a lid is used.

14. A biological sample holder, comprising an elongate generally planar support including a handle end and an opposing biological sample storage end, wherein a substrate for accepting and storing a biological sample is mounted to the holder at said sample storage end.

15. The biological sample holder of claim 14, wherein the substrate is so mounted within a recess in the surface of the support.

16. The biological sample holder of claim 14, wherein the recess has a local surface providing a reaction surface for allowing a sub-sample to be removed from the medium by mechanical action, such as cutting or punching.

17. The biological sample holder of claim 15, wherein the recess includes a window across which the substrate is located, to promote drying of a biological sample on the medium, and to allow a sub-sample of the medium to be removed by accessing both sides of the substrate.

18. The biological sample holder of claim 14, wherein the support includes one or more of: indicia (e.g. a bar code); a radio frequency transponder; and a writable area.

19. The biological sample holder of claim 14, wherein the holder is approximately rectangular in cross section, with rounded edges, having a generally uniform thickness of about 0.5 to 3 mm and a width of about 8 to 10 mm at its widest.

20. The biological sample holder of claim 14, wherein the substrate is sheet material about 25 mm long and about 6 to 7 mm in width.

21. The biological sample holder of claim 14, wherein the substrate is a fibre based paper, for example cellulose fibre paper sold under the trade name of FTA, FTA Elute, or 903, all by Whatman Inc or a silica fibre based paper.

22. A multi-well container holding a plurality of biological sample holders of claim 15.

23. The multi-well container of claim 22, wherein said container comprises an array of wells each having a well which is generally square or rectangular in plan.

24. The multi-well container of claim 23, wherein said holders are supportable lengthwise in a respective well, and the width of the holders is wider than the largest width of the wells, such that each holder is supportable only generally diagonally in its respective well.

25. The multi-well container of claim 22, further comprising a lid for protecting samples stored on the holders and optionally a removable base.

26. The multi-well container of claim 22, further including means for purging said container with gas or dry air to reduce moisture and accelerate drying of samples applied to said sample holders.

27. (canceled)
